(19) United States
(12) Patent Application Publication
(10) Pub. No.: US 2010/0285563 A1
(43) Pub. Date: Nov. 11, 2010

(54) Non-Ribosomal Peptide Synthetases

(76) Inventors: Mohamed A. Marahiel, Marburg (DE); Henning Mootz, New York, NY (US); Dirk Schwarzer, Marburg (DE); Sascha Dokel, Cambridge, MA (US); Uwe Linne, Rosenthal (DE)

Correspondence Address: K&L Gates LLP, STATE STREET FINANCIAL CENTER, One Lincoln Street, BOSTON, MA, 02111-2950, US

(21) Appl. No.: 12/819,751

(22) Filed: Jun. 21, 2010

Related U.S. Application Data
(62) Division of application No. 10/110,330, filed on Dec. 23, 2003.

(30) Foreign Application Priority Data
Oct. 22, 1999 (DE) 19951196.9

(51) Int. Cl. C12N 9/00

(52) U.S. Cl. 435/183

(57) ABSTRACT

Novel tailor-made artificial non-ribosomal peptide synthetases (NRPSs) for non-ribosomal synthesis and/or modification of peptides of a predetermined length and composition and/or for modification of individual amino acids are described. The fusion of building units of said peptide synthetases in particular linker regions makes it possible to specifically prepare by means of “modular molecule construction kits” NRPSs which are capable of synthesizing peptides of a desired structure.

The present invention relates to novel tailor-made non-ribosomal peptide synthetases (NRPSs), to the preparation thereof and to the use of said synthetases for synthesizing known or else artificial, constructed peptides or for modifying individual amino acids.

Non-ribosomal peptide synthetases (NRPSs or peptide synthetases) are modular enzymes having unusual structures and important biological functions. Numerous peptides of pharmaceutical and/or biotechnological interest are synthesized by large enzyme complexes, the “NRPSs” (Marahiel et al. (1997), Chem. Rev. 97: pp. 2651-2673). Said peptides include known medicaments such as cyclosporin A and vancomycin. The wide variety of bioactive peptides synthesized in this way are a result of the great structural variety of NRPSs. NRPSs often incorporate unusual building blocks such as, for example, α-hydroxyamino acids or non-proteinogenic amino acids. The residues may be further modified, for example by N-methylation, heterocyclic ring formation or epimerization. Many of the peptides synthesized by natural NRPS are cyclized via ester or peptide bonds. Generally it can be said that NRPSs play a key part in the synthesis of complex biocompounds.

It has already been found previously that the structure of the multifunctional protein templates is essentially modular. Module denotes the catalytic unit which incorporates a specific basic building block for elongation, i.e. an α-amino acid in most cases, into the product (peptide) (Marahiel et al. (1997), Chem. Rev. 97: pp. 2651-2673). The order of the modules within the NRPS determines the sequence of the building blocks within the product. The individual modules are composed of “domains” which are in each case responsible for a particular reaction step. Thus, for example, the adenylation domain (A domain) determines entering of the substrate into the non-ribosomal peptide synthesis in that the A domain selects and adenylates the substrate, usually an amino acid. The activated amino acid can then be bound via a thioester bond to the cofactor 4′-phosphopantethein of a thiolation domain (T domain) among experts, the T domain is also denoted PCP domain (peptidyl carrier-protein domain). From there, it is possible for the aminoacyl or peptidyl residues to be condensed to the neighboring module. This reaction is catalyzed by the condensation domain (C domain). These three domains, C, A and T, usually form the base unit of multimodular NRPSs, with the first NPRS module normally not containing a C domain. The last NRPS module normally contains a thioesterase or termination domain (Te domain) or, as an alternative, the reductase domain (R domain) which is responsible for the liberation of the synthesis product. The Te domain may catalyze a transfer to a water molecule (hydrolysis, leading to linear products) or to a functional group of the peptide just produced (amide or ester linkage, leading to cyclic or branched cyclic products). Instead of a C domain, it is also possible for a cyclization domain (Cy domain) to be present, which in addition to condensation causes cyclization of the relevant peptide part.

At positions at which a modified amino acid is incorporated into the peptide, modification domains are inserted into the appropriate module. Examples of modification domains are the epimerization domain (E domain), the N-methylation domain (M domain), the N-formylation domain (F domain) and the oxidation domain (Ox domain).

An NRPS may but need not be distributed over more than one enzymic subunit. In this case, normal in systems of bacterial origin, the appropriate subunits interact and pass on the growing peptide chain (the entire tyrocidine NRPS, for example, consists of three subunits, NRPS TycA, TycB and Tyc [lacuna], which contain one, three and six modules, respectively.

There have already been attempts to influence by exchanging amino acids within A domains the specificity of said domain (German patent application No. 19909164.3-44). The exchange of domains in order to vary known compounds was also proposed previously (EP-A-0 789 078).

Such exchanges always had a problem in that there seemed to be no clearly defined borders or transitions between the individual domains (Schneider et al. (1998) Mol. Gen. Genet. 257, pp. 308-318). Moreover, it was found, for example, that C and A domains in each case have selectivities which, when combined artificially, may lead in practice to incompatibilities and stop the entire peptide synthesis (Belshaw et al. (1999), Science 284, pp. 486-489).

We have shown now that linkers of high variability are connected between the individual domains and/or modules in a region of a few amino acids. The linkers tolerate the alterations in the amino acid sequence, caused by specific introduction of cleavage sites for restriction endonucleases at the DNA level, without impairing the function of said domains or modules. The peptide synthetases generated in this way are distinguished by high activity compared with the NRPS generated in a conventional manner or are, for the first time ever, capable of synthesizing the desired peptides.

Likewise, the method described makes it possible to integrate into the NRPS via fusion, modules which incorporate acetate or propionate units into the peptide backbone. These units are derived from coenzyme-A esters of malonic acid or from an α-substituted, for example alkyl-substituted, malonic acid. Said modules are found in polyketide synthases (PKSs) whose structure, like that of NRPS, is modular and for which in principle the same synthesis principle of binding of the intermediates via the thioesters is followed (Staunton et al. (1997), Chem. Rev. 97, pp. 2611-2629). Hybrids of NRPS and PKS modules are also found in natural systems such as the enzymes for yersiniabactin biosynthesis. In this case, a module is composed of the domains ketosynthase (KS) for bond formation (corresponding to the C domain), acyltransferase (AT) for covalent loading of the enzyme with the monomeric building block as thioester (corresponding to the A domain) and an acyl carrier protein (ACP) for binding the building block as thioester to the sulfhydryl group of the cofactor 4′phosphopantethein (corresponding to the T domain). Additional reductive domains correspond to the modification domains in NRPS, which can reduce the β-keto group resulting from condensation in steps to the hydroxyl function (ketoreductase, KR), to the α-β-unsaturated chain (dehydratase, DH) or to the completely saturated chain (enoylreductase, ER). The specificity for acetyl or propionyl building blocks is located in the AT domain which recognizes either malonyl- or methylmalonyl-CoA as substrate.

Due to the fusion of gene fragments coding for single modules or domains in the regions coding for the linkers, for example by specifically introducing defined restriction endonuclease cleavage sites into said linker regions, it was surprisingly possible to synthesize recombinant NRPSs which in turn are capable of synthesizing peptides of a previously defined structure. Thus, a method is described here for the first time, which makes it possible to specifically prepare by means of a “modular molecule construction kit” NRPSs which are to be prepared for synthesizing constructed peptides, preferably those which are known to have or expected to have an advantageous action.

In accordance with the invention, constructed peptides may be known peptides and derivatives thereof and also, preferably, peptides which have been designed, for example, by computer aided molecular design or similar methods.

Preference is also given to a method for peptide biosynthesis, in which, owing to the explanations given here and the technical teaching of the invention, the recombinant gene or recombinant genes which are assembled from gene fragments coding for modules and code for corresponding peptide synthetases of the invention are integrated into a microorganism, preferably Bacillus subtilis, Escherichia coli, Saccharomyces cerevisiae or microorganisms of the genus Streptomyces, for example integrated as vector-encoded genes or into the chromosome, according to methods known per se to the skilled worker, in order to produce the desired peptide synthetases which then in turn can synthesize the desired tailor-made peptide in the microorganism used.

The linker regions for the fusion are preferably located in the following positions:
a) The linker region between a T domain and the subsequent domain, for example a C, E, KS or Te domain, is 12 amino acids in length and is located between amino acids 34 to 45 (in each case inclusively), carboxy-terminally of the 4′-phosphopantethein-binding sites in the sequence DxFFxxLGG(DH)S(IL) (sequence denoted according to Marahiel et al. (1997), Chem. Rev. 97: pp. 2651-2673) of the T domain. The region between amino acids 38 and 39 is particularly preferred for the fusion.
b) The linker region between an A domain and a T domain is 9 amino acids in length and is located in the region of amino acids 10-18 (in each case inclusively), carboxy-terminally of the lysine in the sequence NGK(VL)DR (sequence A10 of the A domain (sequence denoted according to Marahiel et al. (1997), Chem. Rev. 97: pp. 2651-2673)). The region between amino acid 16 and 17 is particularly preferred for the fusion.
c) The linker region between a C domain and an A domain is 23 amino acids in length and is located in the region of amino acids 38-60 (in each case inclusively), amino-terminally of the leucine in the sequence L(TS)YxEL (sequence A1 of the A domain (sequence denoted according to Marahiel et al. (1997), Chem. Rev. 97: pp. 2651-2673)). The region between amino acids 47 and 48 is particularly preferred for the fusion.
d) The linker region between an E and a C domain is 20 amino acids in length and is located in the region of amino acids 9-28 (in each case inclusively), amino-terminally of the serine of the sequence SxAQxR(LM)(WY)xL (sequence C1 of the C domain (sequence denoted according to Marahiel et al. (1997), Chem. Rev. 97: pp. 2651-2673)). The region between amino acids 20 and 21 is particularly preferred for the fusion.

The invention relates to novel NRPSs according to the definition stated in claim 1. The invention further relates to a method for preparing said NRPSs, comprising the measures defined in claim 18.

If polyketide synthases (PKSs) are intended to be incorporated into the NRPSs, further linker regions may be employed for the fusion. These linker regions are preferably located in the following positions:
e) The linker region between a T domain and a KS domain after it is 12 amino acids in length and is located in the region between amino acids 34 to 45 in each case inclusively), carboxy-terminally of the serine in the sequence DxFFxxLGG(DH)S(IL) of the T domain. The region between amino acids 38 and 39 is particularly preferred for the fusion.
f) The linker region between an ACP domain and a domain after it is 12 amino acids in length and is located in the region between amino acids 34 to 45 (in each case inclusively), carboxy-terminally of the 4′-phosphopantethein-binding serine in the sequence (LI)GxDSL of the ACP domain. The region between amino acids 38 and 39 is particularly preferred for the fusion.
g) The linker region between an A domain and an ACP domain may be generated by fusion within the region of amino acids 10-18 (in each case inclusively), carboxy-terminally of the lysine in the sequence NGK(VL)DR of the A domain, and within the region 46-77 amino acids, amino-terminally of the 4′-phosphopantethein-binding serine in the sequence (LI)GxDSL of the ACP domain. The region between amino acids 16 and 17, carboxy-terminally of the lysine in the sequence NGK(VL)DR of the A domain, and also the region 46-77 amino acids, amino-terminally of the 4′-phosphopantethein-binding serine in the sequence (LI)GxDSL of the ACP domain are particularly preferred for the fusion.

Preference is given to incorporating into the DNA region coding for the relevant linkers an artificial cleavage site for one of the following restriction enzyme pairs: Bam HI and Bgl II, and Xba I and Nhe I.

Using said cleavage sites has the advantage that, after ligation, they can no longer be recognized by the restriction enzymes originally used. The preferred enzyme pairs generate compatible cleavage sites which, when ligated into one another, no longer show restriction sequences for the two enzymes.

In principle, however, owing to the variability of said region, there are no restrictions regarding the use of other restriction enzymes.

When modules containing modification domains are excised, the position of these domains should be taken into account. If the modification domain is located between an A domain and a T domain, as is the case, for example, for the methylation domain, there is no need for a change compared with excising a simple CAT module:

C A M T|C A T (cleavage site|).

If, however, the modification domain is located after the T domain of a module, as is the case, for example, for the epimerization domain (E domain), the cyclization domain (Z domain) and the reductase domain (R domain), this should be taken into account when carrying out the fusion in that, in this case, fusion should take place in the linker region between A domain and T domain.

We have shown that the T domain and the subsequent domain influence each other. This means in practice that it is advantageous for an optimal synthesis if, during the fusion process, the function of the domain after the T_{xy} domain corresponds to that of the domain (xy) which also originally followed the T_{xy} domain in their natural structure. Thus, if a C domain followed a T domain, the latter is a T_{c} domain which can in turn be fused in fusion steps to a C domain, for example

C A T|C A T (cleavage site|).

If, however, a modification domain, for example an E domain, is incorporated after the T domain, then the T domain used should advantageously be a T_{E} domain, i.e. a T domain which was followed in its original structure by an E domain, too. In order to ensure this, in such a case the T domain and E domain are advantageously excised together:

C A|T_{E} E C A T (cleavage site|).

In accordance with this, T_{C}, T_{Te}, T_{KS} and T_{Z} domains, on the other hand, are compatible and normally need not be taken into account separately.

We have also shown that the first module of an NRPS subunit starting with a C domain naturally has a variable region of up to 40 amino acids, usually 10 to 15 amino acids, at the front, and this region is important for the activity of the synthetase, in particular for the reaction with the preceding NRPS subunit from which the growing peptide chain is transferred.

Surprisingly, it turned out that individual modification domains, too, fulfill their modification function in constructs which consist of only one module, free or bound to a solid phase. This makes it possible to construct modification generators which convert L-amino acids into D-amino acids, for example by means of an epimerization domain. The skilled worker knows per se the suitable design of such generators from the known enzyme reactors.

The modification module constructs essentially comprise the A and T domains which have affinity for the amino acid to be converted and which are connected to the desired modification domain and, where appropriate, a Te domain or, where appropriate, an R domain. It is possible to add further peptide sequences to the front or the end in order to improve the efficacy of the construct or its manageability, for example attachment to a solid phase.

The modification generators thus are a particular embodiment of the NRPS of the invention or represent application of the inventive method of the fusion of individual domains to an individual module.

The invention relates in particular to a method for tailor-made synthesis of artificial non-ribosomal peptide synthetases (NRPSs), in which a1) a DNA sequence is selected, which codes for a naturally occurring sequence of domains or modules, which can attach a predetermined sequence of amino acids of a predetermined peptide, a2) a predetermined portion which codes for one or more domains or modules which can attach an amino acid or a predetermined sequence of amino acids of the peptide, is removed from said DNA sequence by means of methods known per se, and a3) the remaining DNA part sequences are fused by means of methods known per se to give the desired NRPS in the linker regions described above.

The invention furthermore relates in particular to a method for tailor-made synthesis of artificial non-ribosomal peptide synthetases (NRPSs), in which b1) a DNA sequence is selected, which codes for a naturally occurring sequence of domains or modules, which can attach a predetermined sequence of amino acids of a predetermined peptide, b2) a predetermined portion which codes for one or more domains or modules, which can attach an amino acid or a predetermined sequence of amino acids of the peptide, is removed from said DNA sequence by means of methods known per se, and b3) the remaining DNA part sequences are fused together with a DNA sequence which codes for a domain or a sequence of domains, for a module or a sequence of modules, which can attach a predetermined amino acid or a predetermined sequence of amino acids of a peptide, by means of methods known per se to give the desired NRPS in the linker regions described above.

A variant of the method of the invention is constructing recombinant NRPS for producing a defined peptide by amplifying from various DNA sections coding for NRPS by means of PCR in each case those DNA fragments which code for the required domains and/or modules. In this connection, it is possible to introduce into the oligonucleotides used for the PCR cleavage sites which allow easy linkage of the DNA fragments via ligation. The DNA fragments are selected such that the region of its linkage site codes for the linker regions between domains or modules. Taking into account said linker regions is crucial for the activity of the NRPS newly prepared in this way. The DNA fragments may be incorporated in steps into a suitable vector, for example pTZ18 or pUC18 (Pharmacia, Freiburg, Order. No. 27-4949-01), so that finally the vector contains the fragments coding for the modules or domains, linked to one another in the desired order.

Thus, for example, a plurality of fragments which are provided with the PCR oligonucleotides with cleavage sites for the restriction endonucleases Nhe 1 (5′ end) and Xba 1 (3′ end) can be ligated by proceeding as follows.

Starting from vector pTZ18R, a single cycle comprises
1) hydrolytic cleavage with Xba 1,
2) dephosphorylation with CIP phosphatase and ligation of the purified DNA fragment with
3) the in each case next PCR fragment which had been cleaved hydrolytically with Xba 1 and Nhe 1 by means of T4 DNA ligase;
4) transformation of competent Escherichia coli cells with an aliquot of the ligation mixture;
5) from colonies which were obtained after selection on ampicillin,
6) preparation of extrachromosomal DNA;
7) determination of the desired plasmid construct, i.e. insertion of the DNA fragment into the plasmid in correct orientation, by suitable restriction analyses.

A plasmid obtained in this way can be used for insertion of the next DNA fragment.

The recombinant DNA obtained in this way is then expressed according to methods known per se to the skilled worker in a suitable organism, preferably a microorganism, preferentially in Escherichia coli. Bacillus subtilis, Saccharomyces cervisiae or microorganisms of the genus Streptomyces, where appropriate by using other suitable vectors. The thus produced recombinant NRPS can be modified with the cofactor 4′-phosphopantethein either by coexpressing in said microorganism the gene for a 4′-phosphopantethein transferase (Stachelhaus et al. (1998), J. Biol. Chem. 273: pp. 22773-22781), or, after isolation, the recombinant NRPSs can be modified in vitro using a 4′-phosphopantethein transferase and coenzyme A (Lambalot et al. (1996) Chem. & Biol. 3: pp. 923-926).

The isolation of the expressed enzymes can either be isolated according to methods likewise known per se to the skilled worker and be employed in vitro for synthesis of the desired peptides, or they are left in the organism and the desired peptides are synthesized in vivo. In this way it is also possible to synthesize in vivo in particular pharmacologically active peptides in eukaryotic cells, in particular in mammalian cells and in plant cells.

Thus, the construction may be carried out essentially at the DNA level.

The following examples merely serve to illustrate the invention and are not intended to restrict disclosure thereof in any way. The sequences of the oligonucleotides used in these examples are listed in Table 2 after Example 3.

### EXAMPLE 1

### Preparation of Artificial Peptide Synthetases

A synthetic peptide synthetase was prepared, which synthesizes a peptide product according to the method of the invention. For this purpose, a system consisting of two modules was extended to a system consisting of three modules. The first two modules, TycA and ProCAT, correspond to the first two modules of the tyrocidin peptide synthetases from Bacillus brevis ATCC8185 (Mootz et al. (1997) J. Bacteriol. 179: pp. 6843-6850). TycA contains an A domain which is specific for phenylalanine, a T domain and an E domain which converts L-phenylalanine bound to the T domain into the D form. ProCAT contains a C domain, an A domain which is specific for L proline and a T domain. With addition of ATP (10 mM), MgCl₂ (10 mM), L-phenylalanine and L-proline (je 1 mM) in a suitable buffer system (e.g. assay buffer: HEPES 50 mM, NaCl 100 mM pH 8.0), TycA and ProCAT produced the dipeptide D-Phe-Pro which was bound as a thioester to ProCAT. A subsequent nonenzymically catalyzed reaction led to the removal by cleavage of the cyclic D-Phe-Pro-diketopiperazine. Due to fusion at the genetic level, further modules XaaCAT were then connected with ProCAT according to the method of the invention so that enzymes consisting of two modules, of the type ProCAT-XaaCAT-Te (“-” corresponds to the fusion site), were produced, which had the predicted specificity and, together with TycA, synthesized the predicted tripeptides (Examples 1a and 1b).

### EXAMPLE 1a

The last module of tyrocidin peptide synthetase TycC, LeuCAT, consists of a C domain, an A domain with leucine specificity and a T domain. Immediately thereafter follows a Te domain as the last TycC component. The gene fragment for the module including the Te domain, LeuCATTe, was fused to the fragment coding for ProCAT according to the method of the invention. The enzyme obtained in this way, ProCAT-LeuCATTE (“-” corresponds to the fusion site), was in the aminoacyl adenylate formation reaction specific for the amino acids L-proline and L-leucine. For the aminoacyl adenylate formation reaction the “ATP/PPi exchange reaction was carried out. For this purpose, the amino acids to be assayed and ATP were in each case initially introduced into Eppendorf reaction vessels and preincubated at 37° C. To this, a mixture of enzyme, nonradioactively labeled and radiolabeled PPi and MgCl₂ (in assay buffer), likewise preincubated at 37° C., was added using a pipette. The reaction mixture was incubated at 37° C. for 15 min, then transferred to ice and admixed with 0.5 ml of ice-cold termination solution. The mixture was vortexed and incubated on ice for 1 min. The activated carbon was then pelleted by centrifugation (13,000 rpm; 1 min), washed twice via resuspension in 0.8 ml of H₂O and another centrifugation, resuspended in 0.5 ml of H₂O, transferred into 20 ml scintillation vials and admixed with 4 ml of scintillation fluid Rotiszint Eco Plus. Thus it was possible to measure the count rate of the sample in a scintillation counter.

| | | |
|---|---|---|
| ATP-PPᵢ Exchange | Amino acid 10 mM | 10 μl |
| reaction | ATP 100 mM | 5 μl |
| | Enzyme | 50 pmol |
| | MgCl₂ | 1 μl |
| | Sodium pyrophosphate 50 mM | 0.2 μl |
| | [³²P]-pyrophosphate | 0.15 μCi |
| | assay buffer pH 8.0 | to 100 μl |
| Termination solution | Sodium pyrophosphate | 100 mM |
| | Perchloric acid | 560 mM |
| | Activated carbon (Norit A) | 1.2% (w/v) |

When ProCAT-LeuCATTe was incubated with TycA, it was possible to detect production of the tripeptide D-Phe-Pro-Leu. For this purpose, in each case 50 pmol of TycA and of ProCAT-LeuCATTe were incubated in a total volume of 100 μl in assay buffer containing 10 mM MgCl₂, 5 mM ATP, 1 mM L-Phe, 1 mM L-Pro and 1 mM L-Leu in an Eppendorf reaction vessel at 37° C. for 2 hours. After stopping the enzymic reaction by adding 50 μl of n-butanol/chloroform (4:1), the mixture was concentrated to dryness under reduced pressure. The dry pellet was admixed with 10% HPLC buffer B (0.04% HCOOH in methanol) in HPLC buffer A (0.05% v/v HCOOH in H₂O), dissolved and the clear supernatant after centrifugation was removed. This supernatant was then used for product separation and identification using an HPLC/MS apparatus (solid phase 250/3 Nucleosil-C18 reversed phase column from Macherey & Nagel, liquid phase gradient HPLC buffer A/B: 0 min, 10% HPLC buffer B; 1 min, 30% HPLC buffer B; 20 min, 100% HPLC buffer B; 30 min 100% HPLC buffer B; 35 min, 10% HPLC buffer B; 50 min, 10% HPLC buffer B; flow rate 0.3 ml/min). The product D-Phe-Pro-Leu was detected via its migratory behavior which was identical to that of a chemically synthesized D-Phe-Pro-Leu standard and its mass ([M+H]⁺ peak at 376 Da). The rate of formation of D-Phe-Pro-Leu was determined to 2.1 min⁻¹ (see below for construction of the expression plasmids and isolation of the recombinant NRPSs).

### EXAMPLE 1b

The fifth and second-last module of the tyrocidin peptide synthetase TycC, OrnCAT, consists of a C domain, an A domain with ornithine specificity and a T domain. The gene fragment for this module was fused to the fragment coding for proCAT according to the method of the invention. The thus obtained enzyme ProCAT-OrnCAT (“-” corresponds to the fusion site) was specific for the amino acids L-proline and L-ornithine and produced, when incubated with TycA (as above, additional addition of 1 mM L-ornithine) the tripeptide D-Phe-Pro-Orn. However, the latter was not removed by enzyme-catalyzed cleavage.

In another step, the gene fragment coding for the Te domain of TycC was fused according to the method of the invention such that the enzyme ProCAT-OrnCAT-Te (“-” corresponds to the fusion site) was obtained. This enzyme was incubated with TycA (as above, additional addition of 1 mM L-ornithine) and produced the tripeptide D-Phe-Pro-Orn which was also cleaved off by the enzyme. For this purpose, in each case 50 pmol of TycA and of ProCAT-OrnCAT-Te were incubated in a total volume of 100 μl in assay buffer containing 10 mM MgCl₂, 5 mM ATP, 1 mM L-Phe, 1 mM L-Pro and 1 mM L-Orn for 2 hours in an Eppendorf reaction vessel at 37° C. Stopping of the reaction, working-up and HPLC/MS analysis were carried out as described in Example 1a. The product D-Phe-Pro-Orn was detected via its mass ([M+H]⁺ peak at 377 Da). The rate of formation of D-Phe-Pro-Orn was determined to 0.15 min⁻¹ (see below for construction of the expression plasmids and isolation of the recombinant NRPSs).

### Construction of Expression Plasmids and Isolation of Recombinant NRPSs for Examples 1a and 1b:

All gene fragments were amplified from chromosomal DNA of Bacillus brevis ATCC8185 using Vent polymerase from New England Biolabs (order No. 254S, Schwalbach/Taunus, Germany, used according to manufacturer's instructions). Oligonucleotides Seq ID-NO:1 and Seq ID-NO:2 were used for the TycA-encoding gene fragment, oligonucleotides Seq ID-NO:3 and Seq ID-NO:4 for the ProCAT-encoding gene fragment, oligonucleotides Seq ID-NO:5 and Seq ID-NO:6 for the LeuCATTe-encoding gene fragment, oligonucleotides Seq ID-NO:7 and Seq ID-NO:8 for the OrnCAT-encoding gene fragment and oligonucleotides Seq ID-NO:9 and Seq ID-NO:6 for the Te domain-encoding gene fragment. The PCR amplificates were purified using the QIAquick-spin PCR purification system (Qiagen; Hilden, Germany, catalogue No.: 28104) and their ends were hydrolytically cleaved by restriction endonucleases Nco I and Bam HI (TycA, ProCAT), Bam HI (OrnCAT, LeuCATTe) and Bam HI and Bgl II (Te). The particular restriction endonuclease recognition sequences were contained in the oligonucleotides used (all restriction endonucleases were from Amersham/Buchler; Brunswick, Germany; Nco I: order No. E1160Z; Bam HI: order No. E1010Y and Bgl II: order No. 1021Y). The TycA- and ProCAT-encoding gene fragments were ligated into a vector pQE60 (Qiagen; Hilden, Germany, order No.: 33603), hydrolytically cleaved with Nco I and Bam HI, using T4 DNA ligase (Amersham/Buchler, Brunswick, Germany, E70005Y). After transforming E. coli XL1Blue with the ligation mixture and selecting on solid LB medium using ampicillin (100 μg/ml) as selection antibiotic and subsequently incubating at 37° C. overnight, ampicillin-resistant transformants were obtained. Plasmid DNA was isolated in each case from said transformants and subsequently tested for identity of the desired construct by restriction analysis (using enzymes Nco I, Bam HI, Hind III and Ava I). Thus it was possible to obtain plasmids pTycA and pProCAT plasmid pProCAT was again hydrolytically cleaved by Bam HI, dephosphorylated by CIP phosphatase (New England Biolabs, Schwalbach/Taunus, Germany, order No.: 290S) purified using the QIA quick-spin PCR purification system and used as vector for cloning the hydrolytically cleaved PCR fragments for LeuCATTe and OrnCAT (see above) (ligation by T4 DNA ligase). After transforming E. coli XL1Blue with the ligation mixture and selecting on LB using ampicillin (100 μg/ml), ampicillin-resistant E. coli colonies were obtained. These were used for isolating plasmid DNA. After restriction analysis of said plasmids (using enzymes Nco I, Bam HI, Hind III and Ava I), plasmids pProCAT-LeuCATTe and pProCAT-OrnCAT were obtained. The correct orientation of the LeuCATTe- and OrnCAT-encoding fragments was verified by restriction analysis using restriction endonuclease Hind III (Amersham/Buchler, Brunswick, Germany, order No. E1060Z). Plasmid pProCAT-OrnCAT was again hydrolytically cleaved by Bgl II, dephosphorylated by CIP phosphatase, purified using the QIA quick-spin PCR purification system and used as vector for ligation with the hydrolytically cleaved PCR fragment for the Te domain (see above) (ligation by T4 DNA polymerase). After transforming E. coli XL1Blue with the ligation mixture, selecting ampicillin-resistant transformants on solid LB medium with ampicillin (100 μg/ml), preparing plasmid DNA from said strains and analyzing said plasmids by means of restriction enzymes (using enzymes Nco I, Bam HI, Hind III and Ava I), plasmid pProCAT-OrnCAT-Te was obtained. The correct orientation of the Te domain-encoding fragment was verified by restriction analysis using restriction endonuclease Hind III.

The plasmids obtained were then in each case used for transformation of the E. coli expression strain E. coli BL21/pREP4-gsp (Stachelhaus et al. (1998), J. Biol. Chem. 273: pp. 22773-22781). This strain made possible coexpression with the gsp gene which codes for the 4′-phosphopantethein transferase Gsp which categorizes posttranslational modification of peptide synthetases with the cofactor 4′-phosphopantethein. Vector pREP4-gsp imparts resistance to kanamycin so that transformants were selected using kanamycin (25 μg/ml) and ampicillin (100 μg/ml). The strains thus obtained, BL21/pREP4-gsp/pTycA, BL21/pREP4-gsp/pProCAT, BL21/pREP4-gsp/pProCAT-LeuCATTe, BL21/pREP4-gsp/pProCAT-OrnCAT and BL21/pREP4-gsp/pProCAT-OrnCAT-Te, were used for protein production in liquid medium as described (Stachelhaus et al. (1998), J. Biol. Chem. 273: pp. 22773-22781) and isolated by means of affinity chromatography as described (Stachelhaus et al. (1998), J. Biol. Chem. 273: pp. 22773-22781).

A more detailed description of this procedure may also be found in Example 1c).

### EXAMPLE 1c

### A-T Fusions as Means for Preparing Hybrid Peptide Synthetases

This example describes the use of the fusion site between A and T domains as means for module fusion and for preparing tailor-made hybrid peptide synthetases which synthesize the planned new peptide products.

### Cloning the Hybrid Peptide Synthetase Genes

A 1613 by DNA fragment was amplified from chromosomal DNA of Bacillus licheniformis ATCC 10716 using PCR and the DNA oligonucleotides Seq ID-NO:35 and Seq ID-NO:36. The fragment was purified using the QIA quick spin purification kit and hydrolytically cleaved with the aid of restriction endonucleases Nco I and Pst I (37° C., 16 h).

A 4686 by DNA fragment was amplified from plasmid pTycA (see Example 1a) via PCR using the oligonucleotides Seq ID-NO:37 and Seq ID-NO:38. The amplified fragment contains DNA sequence of the pQE vector and also parts of the tycA gene from Bacillus brevis ATCC 8185. The fragment was purified using the QIA quick spin purification kit and hydrolytically cleaved using restriction endonucleases Nco I and Pst I (37° C., 16 h), followed by a one-hour incubation with alkaline phosphatase at 37° C. Template DNA was then hydrolytically cleaved by incubation with restriction endonuclease Dpn I (37° C., 30 min).

The two fragments described were subsequently ligated using T4-DNA ligase (16° C., 16 h). E. coli XL1 Blue was transformed with a tenth of the ligation mixture (10 μl). Transformants were selected on 2×YT agar plates (ampicillin 100 μg/ml). Plasmid preparations were carried out from 48 transformants which were resistant to ampicillin. 5 transformants contained plasmids of approx. 6.5 kbp in size. The correct insertion of the 1605 bp DNA fragment was confirmed by hydrolytic cleavage of the plasmid DNA by restriction endonucleases and also by terminal sequencing. The plasmid obtained was denoted p[A_{IIe}]_{bacA1}-[TE]_{tycA}.

p[A_{IIe}]_{bacA1}-[TE]_{tycA} was hydrolytically cleaved by hydrolytical cleavage using restriction endonucleases Pst I and Bam HI and separated from the likewise generated 1704 bp DNA fragment by agarose gel electrophoresis. The 5030 bp DNA fragment was isolated, purified by means of the QIA quick spin purification kit and then modified using alkaline phosphatase (37° C., 1 h). A 4131 bp DNA fragment was amplified from chromosomal DNA of Bacillus brevis ATCC 8185 via PCR using the oligonucleotides Seq ID-NO:39 and Seq ID-NO:40. The fragment was hydrolytically cleaved by restriction endonucleases Pst I and Bam HI (37° C., 16 h). Both DNA fragments were ligated [lacuna] T4 ligase (16° C., 16 h). E. coli XL1Blue was transformed with a tenth of the ligation mixture (10 μl). Transformants were selected on 2×YT agar plates (ampicillin 100 μg/ml). Plasmid preparations were carried out from 24 transformants which were resistant to ampicillin. 3 transformants contained plasmids of approx. 9 kbp in size. The correct insertion of the 4125 by DNA fragment was confirmed by hydrolytic cleavage of the plasmid DNA by restriction endonucleases and also by terminal sequencing using pQE standard sequencing oligonucleotides.

The plasmid obtained was denoted p[A_{IIe}]_{bacA1}-[TCA_{Leu}TTe]_{tycC5-6}.

p[A_{IIe}]_{bacA1}-[TCA_{Leu}TTe]_{tycC5-6} was employed for amplifying a 6035 bp DNA fragment via PCR using the oligonucleotides Seq ID-NO:41 and Seq ID-NO:42. The DNA fragment obtained was purified using the QIA quick spin purification kit and hydrolytically cleaved with the aid of restriction endonuclease Pst I (37° C., 16 h). The template plasmid DNA was then hydrolytically cleaved by incubation with Dpn I at 37° C. for 1 hour. The DNA fragment was intramolecularly religated with the aid of T4 ligase. E. coli XL1 Blue were transformed with a tenth of the ligation mixture (10 μl). Transformants were selected on 2×YT agar plates (ampicillin 100 μg/ml). Plasmid preparations were carried out from 24 transformants which were resistant to ampicillin. 12 of those contained the desired plasmid p[A_{IIe}]_{bacA1}-[TCA_{Leu}TTe]_{tycC5-6} (6029 bp), as was shown by hydrolytic cleavage using restriction endonucleases.

p[A_{IIe}]_{bacA1}-[TTe]_{tycC5-6} was hydrolytically cleaved by hydrolytic cleavage using restriction endonucleases Pst I and Hpa I. The 6026 by DNA fragment was purified using the QIA quick spin purification kit and then treated with alkaline phosphatase (37° C., 1 h). A 3117 bp DNA fragment was amplified from chromosomal DNA of Bacillus brevis ATCC 8185 via PCR with the aid of the oligonucleotides Seq ID-NO:43 and Seq ID-NO:44. The DNA fragment was purified using the QIA quick spin purification kit” and then hydrolytically cleaved by restriction endonucleases Pst I and Hpa I (37° C., 16 h). Both DNA fragments were then ligated by T4 ligase. E. coli XL1 Blue was transformed with a tenth of the ligation mixture (10 μl). Transformants were selected on 2×YT agar plates (ampicillin 100 μg/ml). Plasmid preparations were carried out from 24 transformants which were resistant to ampicillin. 2 of those contained a 3117 bp insert, as was shown by hydrolytic cleavage using restriction endonucleases Hpa I and Pst I. The correct insertion was verified by sequencing the fusion sites in the region of the Hpa I and Pst I cleavage sites. The plasmid produced was denoted p[A_{IIe}]_{bacA1}-[TCA_{Phe}]_{tycB2}-[TTe]_{tycC6}.

Expression of the Recombinant Hybrid Genes of p[A_{IIe}]_{bacA1}-[TCA_{Leu}TTe]_{tycC5-6} and p[A_{IIe}]_{bacA1}-[TCA_{Phe}]_{tycB2}-[TTe]_{tycC6}

In each case 1 μl of the constructed plasmids p[A_{IIe}]_{bacA1}-[TCA_{Leu}TTe]_{tycC5-6} and p[A_{IIe}]_{bacA1}-[TCA_{Phe}]_{tycB2}-[TTe]_{tycC6} was used for transforming competent E. coli BL21/pREP4-gsp. Transformants were selected in each case on 2×YT agar plates (ampicillin 100 μl/ml and kanamycin 25 μl/ml). A 5 ml culture of liquid 2×YT medium (ampicillin 100 μl/ml and kanamycin 25 μl/ml) was in each case inoculated with a colony. These cultures were in each case incubated with shaking at 37° C. for 16 h. 1 ml of each culture was used in order to make a glycerol stock of the recombinant strain, which was deep-frozen at −80° C. for storage. 4 ml of each culture were used to inoculate 400 ml of the same medium. The cells were incubated at 30° C. and 25 rpm for 3-4 hours. After they had reached in each case an optical density of 0.7 (OD₆₀₀) transcription of the recombinant genes was induced by adding IPTG (final concentration 200 μM). The cells were in each case cultivated for another 1.5 h and then harvested.

Overproduction of the recombinant proteins p[A_{IIe}]_{BacA1}-[TCA_{Leu}TTe]_{TycC5-6} (protein encoded on plasmid p[A_{IIe}]_{bacA1}-[TCA_{Leu}TTe]_{tycC6} and p[A_{IIe}]_{BacA1}-[TCA_{Phe}]_{TycB2}-[TTe]_{TycC6} (“-” corresponds to the fusion site) (protein encoded on plasmid p[A_{IIe}]_{bacA1}-[TCA_{Phe}]_{tycB2}-[TTe]_{tycC6} was checked by SDS-PAGE by comparing protein samples which had been removed before and after IPTG induction. In the crude extracts of both BL21/pREP4-gsp/p[A_{IIe}]_{bacA1}-[TCA_{Leu}TTe]_{tycC5-6} and BL21/pREP4-gsp/p[A_{IIe]}_{bacA1}-[TCA_{Phe}]_{tycB2}-[TTe]_{tycC6} overproduction of a protein of approx. 215 kDa was observed.

### Purification of Recombinant Proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6}

800 ml cultures of BL21/pREP4-gsp/p[A_{IIe}]_{bacA1}-[TCA_{Leu}TTe]_{tycC5-6} and BL21/pREP4-gsp/p[A_{IIe}]_{bacA1}-[TCA_{Phe}]_{tycB2}-[TTe]_{tycC6} were centrifuged (5000 rpm, 5 minutes) and the cell pellet obtained was then resuspended in 30 ml/l of culture of buffer A (50 mM HEPES, 300 mM NaCl pH 8.0). The cells were used directly or deep-frozen at −20° C. for further use. The cells were disrupted by two passages through a French-Press (working pressure 12,000 PSI). Non-soluble components were then removed by centrifugation at 15,000 rpm for 15 minutes. The clear supernatant was admixed with 1% (v/v) buffer B (50 mM HEPES, 300 mM NaCl, 250 mM imidazole, pH 8.0). The protein solution was subjected to FPLC on an Ni²⁺-NTA-agarose column equilibrated beforehand with 1% buffer B. The flow rate was 0.75 ml/min. After the absorbance (280 nm) had decreased back to the starting level after applying the protein solution, a linearly increasing gradient of buffer B was applied (30 minutes at 30% buffer B, 40 minutes at 100% buffer B). The eluates were collected in 2 ml fractions. The recombinant proteins [A_{IIe}]_{BacA1}-[TCA_{Leu}TTe]_{TycC5-6} and [A_{IIe}]_{BacA1}-[TCA_{Phe}]_{TycB2}-[TTe]_{TyrC6} eluted at buffer B concentrations of approx. 5-10%.

Fractions containing protein [A_{IIe}]_{BacA1}-[TCA_{Leu}TTe]_{TycC5-6} or [A_{IIe}]_{BacA1}-[TCA_{Phe}]_{TycB2}-[TTe]_{TycC6} were detected by means of the Bradford test at 595 nM, combined and dialyzed against assay buffer for 16 h (50 mM HEPES, 100 mM NaCl, 10 mM MgCl₂, 5 mM DTT). The concentration of the dialyzed protein solution was then deter mined once more.

From 1 l of cell culture approx. 5 mg of recombinant proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6}, respectively, were obtained, which were approx. 95% pure, as was possible to verify via STS-PAGE.

### Enzymatic Activity of Recombinant Proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6}

### ATP-PPᵢ Exchange Reaction:

The specificity of the recombinant proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6} with respect to amino acid adenylation was determined indirectly by incorporating ³²P-enabled PP, into ATP during the enzymically catalyzed reverse reaction. The protocol is described in Example 1a.

The protein [A_{IIe}]_{BacA1}-[TCAₗₑᵤTTe]_{TycC5-6} showed specificity for isoleucine and leucine. Valine was also activated at a rate of 30% based on leucine activation. The protein [A_{IIe}]_{BacA1}-[TCA_{Phe}]_{TycB2}-[TTe]_{TycC6} showed tryptophan, phenylalanine and leucine specificity. In this connection, the phenylalanine A domain likewise activated tryptophan.

### Thioester Binding:

The ability to bind the substrate amino acid covalently as thioester was determined via the loading reaction. If the substrate amino acid used is ¹⁴C-labeled, incorporation of radioactivity into precipitated protein may serve as a measure for said loading.

50 pmol of protein [A_{IIe}]_{BacA1}-[TCA_{Leu}TTe]_{TycC5-6} or [A_{IIe}]_{BacA1}-[TCA_{Phe}]_{TycB2}-[TTe]_{TycC6} were incubated with 2 mM ATP, assay buffer and 150 pmol of the particular ¹⁴C-labeled substrate amino acid. After incubation at 37° C. for 10 minutes, proteins were precipitated by adding 10% TCA, pelleted by centrifugation (13,000 rpm, 30 min), and the pellet was washed (1×0.8 ml 10% TCA). The protein pellet was dissolved in 100% formic acid and assayed in a scintillation counter for incorporation of radioactivity.

The proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6} showed in each case a significant activity in thioester binding to the substrate amino acids activated in the ATP-PPᵢ exchange. Protein [A_{IIe}]_{BacA1}-[TCA_{Leu}TTe]_{TycC5-6} was able to bind isoleucine and leucine and protein [A_{IIe}]_{BacA1}-[TCA_{Phe}]_{TycB2}-[TTe]_{TycC6} was able to bind isoleucine and also tryptophan and phenylalanine.

### Product Formation:

Product formation on the enzyme template was carried out by incubating the proteins [A_{IIe}]_{BacA1}-[TCA_{Leu}TTe]_{TycC5-6} and [A_{IIe}]_{BacA1}-[TCA_{Phe}]_{TycB2}-[TTe]_{TycC6} with the particular substrate amino acids (1 mM; see ATP-PPᵢ exchange reaction) and ATP in assay buffer. The reaction products were identified via HPLC and HPLC-MS. The retention times of identified products were compared with chemical standard products (obtained from Bachem, Heidelberg).

50 pmol of protein [A_{IIe}]_{BacA1}-[TCA_{Leu}TTe]_{TycC5-6} or [A_{IIe}]_{BacA1}-[TCA_{Phe}]_{TycB2}-[TTe]_{TycC6} were in each case incubated with 1 mM substrate amino acid, 1 mM ATP in a total assay buffer volume of 100 μl at 37° C. for 2 h. The reactions were in each case stopped by adding 100 μl of butanol. Precipitated proteins were removed using a pipette tip, before in each case the complete solutions were concentrated to dryness in a rotational evaporator under reduced pressure. The residue was taken up in each case in 100 μl of 10% methanol and one tenth of this volume was used for HPLC or HPLC-MS analysis. HPLC analysis was carried out by means of a Hewlett Packard 1100 HPLC system and a Nucleosil C18 3/120 3250 column (Macerey & Nagel, 3×250 mm, pore size 120 angstrom, particle size 3 mm). Detection was carried out using a UV detector at 210 nm. The following gradient was used: 0 min 10% HPLC buffer B; 1 min 30% HPLC buffer B; 20 min 100% HPLC buffer B; 30 min 100% HPLC buffer B; 35 min 10% HPLC buffer B; 50 min 10% HPLC buffer B; the flow rate was 0.35 ml/min (HPLC buffer A: H₂O with 0.05% by volume formic acid, HPLC buffer B: methanol with 0.45% by volume formic acid).

Based on the substrate amino acids isoleucine and leucine, protein [A_{IIe}]_{BacA1}-[TCA_{Leu}TTe]_{TycC5-6} foamed the dipeptide isoleucinyl-leucine at a rate of 0.3 molecules per minute.

Using the substrate amino acids isoleucine and phenylalanine, the protein [A_{IIe}]_{BacA1}-[TCA_{Phe}]_{TycB2}-[TTe]_{TycC6} synthesized the dipeptide isoleucinyl-phenylalanine at a rate of 0.5 per minute. Replacing the substrate amino acid phenylalanine with tryptophan reduced the rate of formation of the corresponding dipeptide isoleucinyl-tryptophan to below 0.02 per minute.

### EXAMPLE 1d

### Role of the T Domain in the Activity of E Domains

This example also serves as an example of modification generators (see Example 3). Artificial fusion proteins of A domains with TE didomains (type A-TE) and of AT didomains with E domains (type AT-E) were prepared.

In this connection, the correctly chosen T domain proved important in order to obtain active proteins. The constructs of type AT_{c}-E were inactive with respect to the epimerization reaction but those of type AT_{E}-E or A-T_{E}E were active (“-” corresponds to the fusion site).

The particular DNA fragments were amplified from chromosomal DNA by PCR using suitable oligonucleotides and firstly cloned intermediately into suitable vectors. In a second cloning step, the vectors were hydrolytically cleaved with the appropriate restriction enzymes and the purified DNA fragments were ligated in the desired manner.

The fragments used (followed, in brackets, by the primers used for amplification) were TE(tycA) (Seq ID-NO:47 and Seq ID NO:48; size of amplificate 1710 bp), E(tycA) (Seq ID_NO:49 and Seq ID_NO:48; 1476 bp), A(tycB2) (Seq ID-NO:50 and Seq ID-NO:51; 1570 bp), AT(tycB2) (Seq ID-NO:50 and Seq ID-NO:52; 1804 bp), A(tycB3) (Seq ID-NO: 53 and Seq ID-NO:54; 1559 bp), AT(tycB3) (Seq ID-NO:53 and Seq ID-NO:55; 1793 bp), A(tycC4) (Seq ID-NO:56 and Seq ID-NO:57; 1570 bp), A(bacA1) (Seq ID-NO: 58 and Seq ID-NO:59; 1613 bp) and AT(bacA1) (Seq ID-NO:58 and Seq ID-NO:60; 1847 bp). All tyc fragments were amplified from chromosomal DNA of B. brevis ATCC8185 and the two bac fragments were amplified from chromosomal DNA of B. licheniformis ATCC 10716 and purified using the QIA quick spin PCR purification kit.

All DNA fragments obtained below after hydrolytic cleaving using restriction endonucleases were purified using the QIA quick spin PCR purification kit. The two E-domain fragments TE(tycA) and E(tycA) were hydrolytically cleaved by restriction endonucleases Bam HI and Bgl II, purified and ligated by means of T4 DNA ligase in each case into pQE60 vectors hydrolytically cleaved by BglII. The remaining fragments were cloned into pQE-60 and pQE70 vectors via the restriction cleavage sites NcoI/BamHI (pQE60) and SphI/BamHI (pQE70). The amplificates were firstly hydrolytically cleaved by the restriction endonucleases stated, the DNA fragments were purified and ligated into the identically treated vectors stated by means of T4 DNA ligase. An aliquot of the ligation mixtures was used in each case for transforming E. coli XL1Blue, the transformants were selected on LB-agar plates (ampicillin 100 μg/ml) and used for plasmid preparation. The correct vector construction was identified by restriction analyses and terminal DNA sequencing. In this way the vectors pQE60-E(tycA), pQE60-TE(tycA), pQE70-A(tycB2), pQE70-AT(tycB2), PQE70-A(tycB3), pQE70-AT(tycB3), pQE60-A(tycC4), pQE60-A(bacA1) and pQE60-AT(bacA1) were obtained.

It was then possible to isolate the DNA fragments of E(tycA) or TE(tycA) from the vectors pQE60-E(tycA) or pQE60-TE(tycA) together with a part of the pQE vector by hydrolytical cleavage using Bgl II and Nde I. The plasmids which carried the fragments coding for the A domains or AT didomains were hydrolytically cleaved by Bam HI and Nde I and, after purification, ligated with the corresponding fragments coding for E(tycA) or TE(tycA) using T4 DNA ligase. After transforming in each case an aliquot of the ligation mixture with E. coli XL1Blue, selecting transformants on LB-agar plates (ampicillin 100 μg/ml and preparing plasmid from the colonies obtained, it was possible to obtain in this way the vector constructs for the artificial hybrid proteins: pQE70-A(tycB2)-TE(tycA), pQE70-AT(tycB2)-E(tycA), pQE70-A(tycB3)-TE(tycA), pQE70-AT(tycB3)-E(tycA), pQE60-A(tycC4)-TE(tycA), pQE60-A(bacA1)-TE(tycA) and pQE60-AT(bacA1)-E(tycA). These expression vectors were used for transformation of E. coli M15/pREP4 and transformants were selected on LB-agar plates (ampicillin 100 μg/ml; kanamycin 25 μg/ml). The strains obtained in this way were then used for protein production as described in Example 1c. Protein purification was carried out likewise in analogy to the method described in Example 1c. After dialysis against assay buffer (100 mM NaCl, 50 mM HEPES, 1 mM EDTA, pH 8.0), the protein solution was divided into aliquots, quick-frozen in liquid nitrogen and stored at −80° C. For the enzyme reactions, in each case a new aliquot was thawed on ice. The proteins obtained were denoted: A(tycB2)-TE(tycA), AT(tycB2)-E(tycA), A(tycB3)-TE(tycA), AT(tycB3)-E(tycA), A(tycC4)-TE(tycA), A(bacA1)-TE(tycA) and AT(bacA1)-E(tycA) (“-” corresponds to the fusion site).

For the enzyme reactions, the enzymes (in each case 100 pmol) were preincubated in assay buffer (total volume 100 μl) containing Sfp (5 pmol, from B. subtilis), MgCl₂ (10 mM and CoA (0.05 mM) at 37° C. for 10 min and thus posttranslationally modified with the cofactor 4′-phosphopanthetein after addition of the particular ¹⁴C-labeled h-amino acid ([¹⁴C]-L-Phe for A(tycB2)-TE(tycA), AT(tycB2)-E(tycA), A(tycB3)-TE(tycA) and AT(tycB3)-E(tycA), [¹⁴-L-Val for A(tycC4)-TE(tycA), [¹⁴C]-L-Ile for A(bacA1)-TE(tycA) and AT(bacA1)-E(tycA)) and ATP (5 mM), the mixture was again incubated at 37° C. for 10 min. The enzymes were precipitated with 1 ml of 10% TCA solution. Incubation on ice for 15 min was followed by centrifugation at 13,000 rpm and 4° C. for 25 min and the supernatant was removed. Washing the pellet twice with 10% TCA (0.8 ml), once with diethyl ether/ethanol (3:1) and once with diethyl ether (in each case 1 ml) was followed by drying in a heating block at 37° C. Addition of 100 μl of 100 mM KOH was followed by incubation in a shaker incubator (14,000 rpm) at 75° C. for 10 min. Addition of 1 ml of methanol was followed by centrifugation at 13,000 rpm/4° C. for 30 min and the supernatant was transferred to a new reaction vessel. After removing the supernatant under reduced pressure, the pellet was resuspended in 20 μl of 50% ethanol and in each case 10 μl were applied to HPTCL ready-made plates CHIR (Merck, Darmstadt). The thin layer chromatographies were developed with acetonitrile/methanol/water (4:1:1). The substances were identified by autoradiography.

After the reaction, it was possible to detect an epimerization activity via the mixture of D- and L-amino acids (R_{f} values: L-Phe 0.6; D-Phe 0.47; L-Ile 0.55; D-Ile 0.48; L-Val 0.52; D-Val 0.42). The two enzymes AT(tycB2)-E(tycA) and AT(bacA1)-E(tycA) having a T domain which is naturally located N-terminally in front of a C domain, i.e. a T_{c} domain, showed no epimerization activity (Type At_{c}-E), whereas in the case of AT(tycB3)-E(tycA) an epimerization reaction was observed (Type AT_{E}-E). In the latter case, the T domain is also naturally located in front of an E domain and is thus a “T_{E} domain”. However, a fusion in front of the T_{C} domain, i.e. connecting the A domain with the T_{E} domain which is followed by an E domain, resulted in epimerization of the activated amino acid by the hybrid enzymes A(tycB2)-TE(tycA), A(tycB3)-TE(tycA), A(bacA1)-TE(tycA) and AA(tycC4)-TE(tycA) (Type A-T_{E}E), too.

### EXAMPLE 2a

### Construction of Hybrid Peptide Synthetases for Production of a Known Peptide Antibiotic

This example illustrates the construction of three peptide synthetases Pa, Pb and Pc according to the method of the invention from gene fragments of various peptide synthetase genes, which synthetases together catalyze synthesis of the peptide skeleton of the lipopeptide antibiotic plipastatin (Tsuge et al. (1996), Arch. Microbiol. 165:243-251). The complete plipastatin is obtained when a cell extract from Bacillus subtilis JH642 is used in addition to all substrates (ATP, MgCl₂ and amino acids, see below), which is required as donor of the β-hydroxy fatty acid. If, however, the modified peptide synthetase Pa2 is incubated with Pb and Pc, a fatty acid-free variant of plipastatin is produced instead.

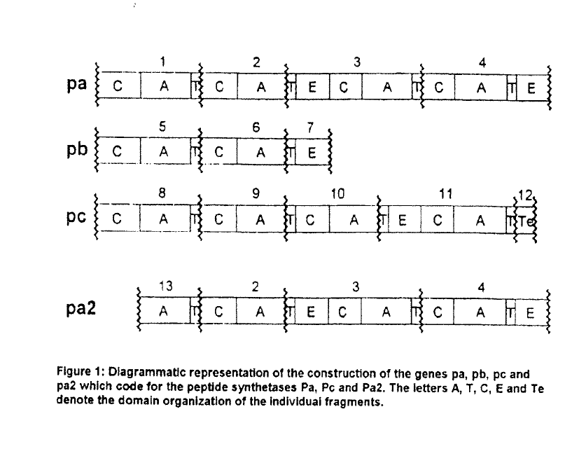

FIG. 1 depicts a diagrammatic representation of the structure of the genes pa, pb, pc and pa2, which code for the peptide synthetases Pa, Pb, Pc and Pa2. The letters A, T, C, E and Te denote the domain organization of the individual fragments. The numbering of the fragments is in line with the numbering used in Table 1 further below.

The recombinant peptide synthetases for production of the antibiotic plipastatin were constructed by using fragments from other genes coding for peptide synthetases and by preparing in steps 3 plasmids (pPa, pPb and pPc). Starting from vector pTZ18R (Pharmacia, Germany), in a single cycle 1) said vector is hydrolytically cleaved by Xba I (Amersham/Buchler, Brunswick, Germany, order No.: R1093Y) 2) dephosphorylated by CIP phosphatase, and the purified DNA fragment is 3) ligated by means of T4 DNA ligase with the in each case next PCR fragment which has been hydrolytically cleaved by Xba I and Nhe I (Amersham/Buchler, Brunswick, Germany, order No.: E1162Y) before hand. 4) Competent Escherichia coli cells are transformed with an aliquot of the ligation mixture and 5) from colonies obtained after selection on ampicillin 6) extrachromosomal DNA is prepared. 7) The desired plasmid construct, i.e. insertion of the DNA fragment into the plasmid in correct orientation, is determined by suitable restriction analyses. A plasmid obtained in this way can be used for insertion of the next DNA fragment.

TABLE 1
Oligonucleotides and origin of the chromosomal
DNA for PCR amplification of the required DNA fragments

| Fragment | Oligonucleotides | |
|---|---|---|
| No. | Seq ID-NO: | From chromosomal DNA of strain |
| 1 | 10 & 11 | Bacillus subtilis JH642 |
| 2 | 13 & 14 | Bacillus brevis ATCC8185 |
| 3 | 15 & 16 | Amycolatopsis orientalis |
| 4 | 17 & 18 | Bacillus subtilis JH642 |
| 5 | 19 & 20 | Bacillus subtilis JH642 |
| 6 | 21 & 22 | Bacillus brevis ATCC8185 |
| 7 | 23 & 24 | Bacillus brevis ATCC8185 |
| 8 | 25 & 26 | Bacillus brevis ATCC8185 |
| 9 | 27 & 28 | Bacillus licheniformis ATCC10716 |
| 10 | 29 & 30 | Bacillus brevis ATCC8185 |
| 11 | 31 & 32 | Bacillus licheniformis ATCC10716 |
| 12 | 33 & 34 | Bacillus subtilis JH642 |
| 13 | 12 & 11 | Bacillus subtilis JH642 |

In the case of pPa, the procedure described is carried out in steps using the DNA fragments 1, 2, 3, 4 and 5. In the case of pPa2, the procedure described is carried out in steps using the DNA fragments 13, 2, 3, 4 and 5. In the case of pPb, the procedure described is carried out in steps using the DNA fragments 5, 6 and 7. In the case of pPc, the procedure described is carried out in steps using the DNA fragments 8, 9, 10, 11 and 12. See Table 1 for the oligonucleotides and chromosomal DNA to be used in each case. See FIG. 1 for a diagrammatic representation of the recombinant NRPS genes constructed in this way.

The plasmids pPa, pPa2, pPb and pPc are then in each case transformed into a suitable E. coli expression strain, for example E. coli BL21/pREP4-gsp (Stachelhaus et al. (1998), J. Biol. Chem. 273: pp. 22773-22781). This strain makes possible coexpression with the gsp gene which codes for a 4′-phosphopantethein tranferase which catalyzes postranslational modification of peptide sythetases with the cofactor 4′-phoshopantethein. The vector pREP4-gsp imparts resistance to kanamycin so that transformants are selected using kanamycin (25 μg/ml) and ampicillin (100 μg/ml). The strains obtained in this way, BL21/pREP4-gsp/pPa, BL21/pREP4-gsp/pPb and BL21/pREP4-gsp/pPc, are used for protein production in liquid medium as described, (Stachelhaus et al. (1998), J. Biol. Chem. 273: pp. 22773-22781). The recombinant proteins Pa, Pa2, Pb and Pc can be purified by common techniques known to the skilled worker.

Another possibility of obtaining 4′-phosphopantethein-modified peptide synthetases is in-vitro modification by adding 4-phosphopantethein transferase Sfp, coenzyme A and MGCl₂ (Lambalot et al. (1996) Chem. & Biol. 3: pp. 923-936).

The purified proteins Pa, Pb and Pc are incubated in equimolar amounts (e.g. 500 mM each) with ATP (10 mM), MgCl₂ (10 mM) and all substrate amino acids (glutamate, ornithine, tyrosine, allo-threonine, valine, proline and isoleucine, all 1 mM) in a suitable buffer (e.g. HEPES 50 mM, 100 mM NaCl, pH 8.0) at 37° C. Addition of a fraction which is obtained by gel filtration of a crude cell extract of Bacillus subtilis ATCC 21332 and contains proteins of a molecular weight of approx. 40 kDa (Menkhaus et al. (1993) J. Biol. Chem. 268: pp. 7678-7684) can initiate synthesis of the lipopeptide plipastatin.

A self-initiating system is obtained by incubating the purified proteins Pa2, Pb and Pc in equimolar amounts with all substrates. These three proteins synthesize the fatty acid-free variant of plipastatin.

### EXAMPLE 2b

As an alternative to in-vitro synthesis of plipastatin according to the method described in Example 2a using the constructed peptide synthetases Pa, Pb and Pc, the fragments coding for Pa, Pb and Pc may be chromosomally integrated into a suitable microorganism by means of homologous recombination, in order to sythesize the antibiotic by the thus constructuted strain. A suitable host strain which may be used is in particular Bacillus subtilis ATCC21332. Since the gene fragments to be integrated partly comprise the gene fragments coding for surfactin peptide synthetases and, putatively, the gene fragments coding for fengycin peptide synthetases, said gene fragments have to be deleted first in Bacillus subtilis ATCC 21332 for controlled integration. This may be carried out in each case using methods known to the skilled worker by cloning 5′- and 3′-flanking regions of the srfAA (surfactin biosynthesis) and pps (or fen, fengycin biosynthesis) operon into a plasmid and cloning between said fragments a common antibiotic resistance cassette which imparts resistance to, for example, erythromycin, spectinomycin, kanamycin or chloramphenicol (for this method, cf. e.g. Schneider et al. (1998) Mol. Gen. Genet. 257: pp. 308-318). Care must be taken here that the comS gene which is essential for the development of Bacillus subtilis competence is located within the srfAA biosynthesis operon (Schneider et al. (1998) Mol. Gen. Genet. 257: pp. 308-318). This gene must therefore be integrated in a first step into the Bacillus subtilis chromosome as a further copy, for example into the amyE gene.

The gene fragments coding for Pa, Pb and Pc are ideally integrated in three steps, for example at the original locations of the srfAA or pps (or fen) operon, using the particular promoters. For stable integration by means of double crossover, 5′- and 3′-homologous sequences must in addition always be present between the plasmid-coded gene fragments for Pa, Pb and Pc and the Bacillus subtilis chromosome. A positive selection for integration of the gene fragment may be carried out by using common gene cassettes which impart resistances to antibiotics such as, for example, erythromycin, spectinomycin, kanamycin or chloramphenicol.

Chromosomal integration, for example into Bacillus subtilis ATCC 21332, of the gene fragments coding for Pa, Pb and Pc, organized in an operon under the control of a suitable promoter, for example the srfAA promotor, leads to in-vivo production of the three peptide synthetases Pa, Pb and Pc which are modified by the Bacillus subtilis ATCC 21332 protein Sfp with the cofactor 4′-phosphopantethein and then synthesize plipastatin.

### EXAMPLE 2c

### Construction of an NRPS-PKS Hybrid System

In this example, the proline-incorporating module ProCAT (see example 1a) is fused to the propionate-incorporating module 6 of 6-deoxyerthronolide B synthase (DEBS1-3, encoded by the genes eryAI, eryAII and eryAIII; DEBS3 contains the modules 5 and 6 (Staunton et al. (1997), Chem. Rev. 97, pp. 2611-2629)). According to the method of the invention, the fusion site is located at amino acids 38 and 39 after the conserved serine in the T domain of ProCAT. The module DEBS3_{—}6 is generated in the same way by choosing the fusion site of amino acids 38 and 39 after the ACP domain of the subsequent module 5. The module DEN3_{—}6 contains a terminal Te domain which serves to remove the product by cleavage.

An expression plasmid pProCAT-DEBS3_{—}6 is constructed by starting from pQE60-ProCAT (see example 1a). A fragment of 5136 bp in size is amplified from chromosomal DNA of Saccharopolyspora erythraea via PCR using the oligonucleotides Seq ID No. 45 and Seq ID No. 46. The amplificate is purified using the Qia Quick spin purification kit and then terminally treated using restriction endonucleases Bgl II and Bam HI. After another purification, this fragment is ligated by T4 DNA ligase with plasmid pQE60-ProCAT which has been hydrolytically cleaved by Bam HI, dephosphorylated by alkaline phosphatase and purified beforehand. An aliquot of the ligation mixture is used for transformation of E. coli XL1 Blue. Transformants are selected on LB-agar plates (100 μg/ml ampicillin). Ampicillin-resistant colonies are used for plasmid preparation. The desired plasmid constructs are identified by restriction analyses using restriction endonucleases. The expression vector prepared in this way, pProCAT-DEBS3_{—}6 (“-” corresponds to the fusion site), is used for transformation of E. coli BL21-pREP4-gsp. Transformants are selected on LB agar plates (100 μg/ml ampicillin; 25 μg/ml kanamycin) and furthermore used for producing the recombinant protein ProCAT-DEBS3_{—}6. ProCAT-DEB3_{—}6 is produced and purified in analogy to the examples 1a-c.

The product is formed by incubating 50 pmol of the enzyme ProCAT-DEBS3_{—}6 with 50 pmol of TycA (see example 1a) in a suitable buffer in a total volume of 100 μl. The reaction mix likewise contains: 1 mM L-phenylalanine, 1 mM L-proline, 0.1 mM (DL) methylmalonyl-CoA, 0.1 mM NADPH, 10 mM MgCl₂ and 5 mM ATP. The reaction mix is incubated at 30° C. for 2 h, then stopped by addition of 50 μl of butanol and concentrated to dryness under reduced pressure. The pellet is taken up in 10% methanol and used in this form for product analysis by means of HPLC/MS. The enzyme system TycA plus ProCAT-DEBS3_{—}6 synthesizes the product 3-{(2S)-1-[(2R)-2-amino-3-phenylpropanoyl]-tetrahydro-1H-2-pyrrolyl}-3-hydroxy-2-methylpropanoic acid.

### EXAMPLE 3

### Modification Generator

The method of the invention also makes possible construction of a monomodular peptide synthetase which recognizes a specific L-amino acid, activates it, converts it into the D-configuration and finally removes it by cleavage as free D-amino acid.

For this purpose, a peptide synthetase gene fragment coding for A, T and E domains is fused to the fragment coding for a Te domain. The plasmid pTycA described in Example 1a is hydrolytically cleaved by Bam HI, the resulting free DNA ends are dephosphorylated (by CIP phosphatase) and this DNA fragment is ligated with the Te domain PCR amplificate (see Example 1b) which has been hydrolytically cleaved beforehand by Bam HI and Bgl II, as described above. The plasmid thus obtained, pTycA-Te (“-” corresponds to the fusion site), is used as described in Example 1a for expressing the peptide synthetase TcyA-Te which recognizes, activates and racemizes free L-phenylalanine and finally removes it by cleavage as D-phenylalanine.

As an alternative to the fragment of the tyrocidine biosynthesis genes, which code for the Te domain, it is also possible to use other fragments coding for Te domains, in particular those which follow an E domain in those peptide synthetases from whose genes they are obtained (e.g. ACV synthetases).

In order to convert other amino acids from the L into the D form, it is possible according to the method of the invention to use A domains of a different amino acid specificity together with a T, E and Te domain.

TABLE 2
Oligonucleotides used in Examples 1-3 (all
from MWGBiotech, Ebersberg, Germany)

| Seq | |
|---|---|
| ID | |
| No. | Oligonucleotide sequence 5′-...3′- |
| 1 | ATACCATGGT AGCAAATCAG GCCAATC |
| 2 | TATGGATCCG CGCAGTCTAT TTGCAAG |
| 3 | TATCCATGGG TGTATTTAGC AAAGAACAAG TTC |
| 4 | TATGGATCCT TCCACATACG CTGCCAG |
| 5 | ATAGGATCCG CCAAAGGGAA TGTCTTCTCG |
| 6 | ATAGGATCCT TTCAGGATGA ACAGTTCTTG |
| 7 | ATAGGATCCG CATTCGAGCA GTTCGAG |
| 8 | ATAGGATCCT TCGATGAACG CCGCCAG |
| 9 | ATAAGATCTC ATAAGCGCTT TGAGAGCAG |
| 10 | ATAGCTAGCG GAGGATCACA TGGAAATAAC TTTTTACCCT |
| 11 | ATATCTAGAA TCAGCTTCCT CTGCAAG |
| 12 | ATAGCTAGCG AGAAAGAGAA GCTGCTTG |
| 13 | ATAGCTAGCG CATTCGAGCA GTTCGAG |
| 14 | ATATCTAGAT CTCAGCATGG TGACATC |
| 15 | ATAGCTAGCC GCGAACCGCG CACCGA |
| 16 | ATATCTAGAT TTGACCATGG CCGCCAG |
| 17 | ATAGCTAGCA AAGAACACGC TTTTACACAG |
| 18 | ATATCTAGAT CCTCTCCTAT AGTTTGTTG |
| 19 | ATAGCTAGCG GAGATTCACT GATGCCG |
| 20 | ATATCTAGAA CGGATAACGG TAGCCAG |
| 21 | ATAGCTAGCG GAAAAGAGAC GTATGTGC |
| 22 | ATATCTAGAT GTCGTCCGCT CGCCGG |
| 23 | ATAGCTAGCG CTGCCTACCA TCCTCC |
| 24 | ATATCTAGAC AACATCTGTT TAGCGCAG |
| 25 | ATAGCTAGCG GAGGTGCCGT AATGAG |
| 26 | ATATCTAGAT TCCACATACG CTGCCAG |
| 27 | ATAGCTAGCC GAAGGAAATG CTTTTACATC |
| 28 | ATATCTAGAG ACATTTACAT TGCCGTCG |
| 29 | ATAGCTAGCG TGTATGTGGC GCCGGG |
| 30 | ATATCTAGAG GTTACGGGCT TGCCTTC |
| 31 | ATAGCTAGCG GAACCGGGTA CGATCC |
| 32 | ATATCTAGAT AATACAAAAC GCGCTAATG |
| 33 | ATAGCTAGCA AACAGCTGAC AGCAGCAA |
| 34 | ATATCTAGAT CCGCTTTATC GTTTGTGC |
| 35 | TTTCCATGGC TAAACATTCA TTAGA |
| 36 | TTCCTGCAGC GCCCCCGCCG TTCTG |
| 37 | AGCCTGCAGG CCTACCATCC TCCGAG |
| 38 | TGGACCCATG GTAATTTCTC CTCT |
| 39 | ATACTGCAGG AGTATGTAGC GCCGC |
| 40 | TATGGATCCT TTCAGGATGA ACAGTTCTTG |
| 41 | ACCGTTAACG AATACGTGGC CCCGAG |
| 42 | AATGTTAACC TCCTGCAGCG CCCC |
| 43 | ACGCTGCAGG ATTACGTCGC CCCGA |
| 44 | AGCGTTAACT GTTGCAGGCT TTCCTTC |
| 45 | TATCCATGGG AAGATCTCTC GTCGGCGCAG CAGAG |
| 46 | ATAGGATCCT GAATTCCCTC CGCCCA |
| 47 | TAAAGATCTG CCTACCATCC TCCG |
| 48 | TATGGATCCG CGCAGTGTAT TTGCAAG |
| 49 | ATAAGATCTA GAAAAAGCGA TCAGGGCATC |
| 50 | AATGCATGCT GACTGCGCAT GAG |
| 51 | TAAGGATCCT GTTGCAGGCT TTCC |
| 52 | ATAGGATCCT TCGATCAAGC GGGCCAAGTC |
| 53 | AAAGCATGCT GACAGCAGCA G |
| 54 | AAAGGATCCC CGGTTCTCCT CCTGGTT |
| 55 | AAAGGATCCC GGGATGACGC GCAGAG |
| 56 | AATCCATGGT CAGCGAGGAA GAGCG |
| 57 | AAAGGATCCT GTCGTCCGCT CG |
| 58 | AAACCATGGT TGCTAAACAT TCATTAG |
| 59 | AAAGGATCCC GCCCCGCCGT TCTG |
| 60 | AAAGGATCCT TCGATAAAAG CGCTC |

1-33. (canceled)

34. A method of synthesizing an artificial non-ribosomal peptide synthetase (NRPS) comprising:
obtaining a first gene fragment from a gene encoding a naturally occurring peptide synthetase, the gene fragment encoding an NRPS thiolation (T) domain;
obtaining a second gene fragment from a gene encoding a naturally occurring peptide synthetase, the gene fragment encoding an NRPS condensation (C) domain;
fusing, in a recombinant DNA plasmid, the second gene fragment to the first gene fragment in a region of the first gene fragment encoding amino acids 34-45 positions carboxy-terminal to the 4′-phophopantethein serine in the sequence DxFFxxLGG(DH)S(IL) of the T domain.

35. The method of claim 34, wherein the first gene fragment encodes an NRPS adenylation (A) domain upstream from the T domain and an NRPS C domain upstream from the A domain.

36. The method of claim 35, wherein the second gene fragment encodes an NRPS A domain downstream from the C domain and an NRPS T domain downstream from the A domain.

37. The method of claim 36, wherein the second gene fragment further encodes a Termination (Te) domain downstream from the T domain.

38. The method of claim 34, wherein a restriction endonuclease site is introduced in the region of the first gene fragment encoding amino acids 34-45 positions carboxy-terminal to the 4′-phosphopantethein serine in the sequence DxFFxxLGG(DH)S(IL) of the T domain.

39. The method of claim 34, further comprising the step of expressing the artificial non-ribosomal peptide synthetase encoded by the recombinant DNA plasmid.

40. The method of claim 34, wherein the first gene fragment is from a first gene and the second gene fragment is from a second gene.

41. The method of claim 34, wherein the first gene fragment is from a first gene and the second gene fragment is from the first gene.

42. The method of claim 34, wherein the second gene fragment is fused to the first gene fragment in a region of the first gene fragment encoding amino acids 38-39 positions carboxy-terminal to the 4′-phosphopantethein serine in the sequence DxFFxxLGG(DH)S(IL) of the T domain to form a DNA plasmid.

43. The method of claim 34, wherein the recombinant DNA plasmid is extrachromosomal.

44. The method of claim 42, wherein the recombinant DNA plasmid is incorporated by a competent host cell.

45. The method of claim 44, wherein the host cell is E. coli.


SEQUENCE LISTING (60 sequences)

SEQ ID NO: 1 - DNA, 27 bp, Bacillus brevis
ataccatggtagcaaatcaggccaatc

SEQ ID NO: 2 - DNA, 27 bp, Bacillus brevis
tatggatccgcgcagtctatttgcaag

SEQ ID NO: 3 - DNA, 33 bp, Bacillus brevis
tatccatgggtgtatttagcaaagaacaagttc

SEQ ID NO: 4 - DNA, 27 bp, Bacillus brevis
tatggatccttccacatacgctgccag

SEQ ID NO: 5 - DNA, 30 bp, Bacillus brevis
ataggatccgccaaagggaatgtcttctcg

SEQ ID NO: 6 - DNA, 30 bp, Bacillus brevis
ataggatcctttcaggatgaacagttcttg

SEQ ID NO: 7 - DNA, 27 bp, Bacillus brevis
ataggatccgcattcgagcagttcgag

SEQ ID NO: 8 - DNA, 27 bp, Bacillus brevis
ataggatccttcgatgaacgccgccag

SEQ ID NO: 9 - DNA, 29 bp, Bacillus brevis
ataagatctcataagcgctttgagagcag

SEQ ID NO: 10 - DNA, 40 bp, Bacillus subtilis
atagctagcggaggatcacatggaaataactttttaccct

SEQ ID NO: 11 - DNA, 27 bp, Bacillus subtilis
atatctagaatcagcttcctctgcaag

SEQ ID NO: 12 - DNA, 28 bp, Bacillus subtilis
atagctagcgagaaagagaagctgcttg

SEQ ID NO: 13 - DNA, 27 bp, Bacillus brevis
atagctagcgcattcgagcagttcgag

SEQ ID NO: 14 - DNA, 27 bp, Bacillus brevis
atatctagatctcagcatggtgacatc

SEQ ID NO: 15 - DNA, 26 bp, Amycolatopsis orientalis
atagctagccgcgaaccgcgcaccga

SEQ ID NO: 16 - DNA, 27 bp, Amycolatopsis orientalis
atatctagatttgaccatggccgccag

SEQ ID NO: 17 - DNA, 30 bp, Bacillus subtilis
atagctagcaaagaacacgcttttacacag

SEQ ID NO: 18 - DNA, 29 bp, Bacillus subtilis
atatctagatcctctcctatagtttgttg

SEQ ID NO: 19 - DNA, 27 bp, Bacillus subtilis
atagctagcggagattcactgatgccg

SEQ ID NO: 20 - DNA, 27 bp, Bacillus subtilis
atatctagaacggataacggtagccag

SEQ ID NO: 21 - DNA, 28 bp, Bacillus brevis
atagctagcggaaaagagacgtatgtgc

SEQ ID NO: 22 - DNA, 26 bp, Bacillus brevis
atatctagatgtcgtccgctcgccgg

SEQ ID NO: 23 - DNA, 26 bp, Bacillus brevis
atagctagcgctgcctaccatcctcc

SEQ ID NO: 24 - DNA, 28 bp, Bacillus brevis
atatctagacaacatctgtttagcgcag

SEQ ID NO: 25 - DNA, 26 bp, Bacillus brevis
atagctagcggaggtgccgtaatgag

SEQ ID NO: 26 - DNA, 27 bp, Bacillus brevis
atatctagattccacatacgctgccag

SEQ ID NO: 27 - DNA, 30 bp, Bacillus licheniformis
atagctagccgaaggaaatgcttttacatc

SEQ ID NO: 28 - DNA, 28 bp, Bacillus licheniformis
atatctagagacatttacattgccgtcg

SEQ ID NO: 29 - DNA, 26 bp, Bacillus brevis
atagctagcgtgtatgtggcgccgcg

SEQ ID NO: 30 - DNA, 27 bp, Bacillus brevis
atatctagaggttacgggcttgccttc

SEQ ID NO: 31 - DNA, 26 bp, Bacillus licheniformis
atagctagcggaaccgggtacgatcc

SEQ ID NO: 32 - DNA, 29 bp, Bacillus licheniformis
atatctagataatacaaaacgcgctaatg

SEQ ID NO: 33 - DNA, 28 bp, Bacillus subtilis
atagctagcaaacagctgacagcagcaa

SEQ ID NO: 34 - DNA, 28 bp, Bacillus subtilis
atatctagatccgctttatcgtttgtgc

SEQ ID NO: 35 - DNA, 25 bp, Bacillus licheniformis
tttccatggctaaacattcattaga

SEQ ID NO: 36 - DNA, 25 bp, Bacillus licheniformis
ttcctgcagcgcccccgccgttctg

SEQ ID NO: 37 - DNA, 26 bp, Bacillus brevis
agcctgcaggcctaccatcctccgag

SEQ ID NO: 38 - DNA, 24 bp, Bacillus brevis
tggacccatggtaatttctcctct

SEQ ID NO: 39 - DNA, 25 bp, Bacillus brevis
atactgcaggagtatgtagcgccgc

SEQ ID NO: 40 - DNA, 30 bp, Bacillus brevis
tatggatcctttcaggatgaacagttcttg

SEQ ID NO: 41 - DNA, 26 bp, Bacillus brevis
accgttaacgaatacgtggccccgag

SEQ ID NO: 42 - DNA, 24 bp, Bacillus brevis und licheniformis
aatgttaacctcctgcagcgcccc

SEQ ID NO: 43 - DNA, 25 bp, Bacillus brevis
acgctgcaggattacgtcgccccga

SEQ ID NO: 44 - DNA, 27 bp, Bacillus brevis
agcgttaactgttgcaggctttccttc

SEQ ID NO: 45 - DNA, 35 bp, Saccharopolyspora erythraea
tatccatgggaagatctctcgtcggcgcagcagag

SEQ ID NO: 46 - DNA, 26 bp, Saccharopolyspora erythraea
ataggatcctgaattccctccgccca

SEQ ID NO: 47 - DNA, 24 bp, Bacillus brevis
taaagatctgcctaccatcctccg

SEQ ID NO: 48 - DNA, 27 bp, Bacillus brevis
tatggatccgcgcagtgtatttgcaag

SEQ ID NO: 49 - DNA, 30 bp, Bacillus brevis
ataagatctagaaaaagcgatcagggcatc

SEQ ID NO: 50 - DNA, 23 bp, Bacillus brevis
aatgcatgctgactgcgcatgag

SEQ ID NO: 51 - DNA, 24 bp, Bacillus brevis
taaggatcctgttgcaggctttcc

SEQ ID NO: 52 - DNA, 30 bp, Bacillus brevis
ataggatccttcgatcaagcgggccaagtc

SEQ ID NO: 53 - DNA, 21 bp, Bacillus brevis
aaagcatgctgacagcagcag

SEQ ID NO: 54 - DNA, 27 bp, Bacillus brevis
aaaggatccccggttctcctcctggtt

SEQ ID NO: 55 - DNA, 26 bp, Bacillus brevis
aaaggatcccgggatgacgcgcagag

SEQ ID NO: 56 - DNA, 25 bp, Bacillus brevis
aatccatggtcagcgaggaagagcg

SEQ ID NO: 57 - DNA, 22 bp, Bacillus brevis
aaaggatcctgtcgtccgctcg

SEQ ID NO: 58 - DNA, 27 bp, Bacillus licheniformis
aaaccatggttgctaaacattcattag

SEQ ID NO: 59 - DNA, 24 bp, Bacillus licheniformis
aaaggatcccgccccgccgttctg

SEQ ID NO: 60 - DNA, 25 bp, Bacillus licheniformis
aaaggatccttcgataaaagcgctc